(19) United States
(12) Patent Application Publication
(10) Pub. No.: US 2021/0299625 A1
(43) Pub. Date: Sep. 30, 2021

(54) Microparticle for Bioanalytical Investigations and Method for Producing Such a Microparticle

(71) Applicant: AVA Lifescience GmbH, Denzlingen (DE)

(72) Inventors: Marcus Dühren- von Minden, Muellheim (DE); Holger Klapproth, Freiburg im Breisgau (DE)

(21) Appl. No.: 17/211,139

(22) Filed: Mar. 24, 2021

(30) Foreign Application Priority Data
Mar. 24, 2020 (DE) 10 2020 001 916.1

(51) Int. Cl. B01J 13/02; G01N 33/543

(52) U.S. Cl. CPC B01J 13/025; G01N 33/5432

(57) ABSTRACT

The invention relaters to a microparticle for bioanalytical investigations and a method for its production. The microparticle is suitable for being transported through a flow cell of a flow cytometer in a fluid stream. The microparticle has a solid particle core and at least one nucleic acid sense biomolecule that is binding-specific for an antisense biomolecule. A layer composed of a water-soluble conjugate is arranged on the particle core, which layer includes at least one linear polymer and/or copolymer, which has at least one first group, by means of which the polymer and/or copolymer can be cross-linked by means of irradiation with an optical radiation having a discrete wavelength, has at least one second group, to which the at least one nucleic acid sense biomolecule is conjugated, and has at least one third group that is bound to the surface of the particle core by way of a functional linker group, wherein the linker group is not capable of binding to the at least one nucleic acid sense biomolecule.

### CROSS-REFERENCE TO RELATED APPLICATION

This application claims priority to German Patent Application No. 10 2020 001 916.1 filed Mar. 24, 2020, the disclosure of which is hereby incorporated by reference in its entirety.

### BACKGROUND OF THE INVENTION

### Field of the Invention

The invention relates to a microparticle for bioanalytical investigations, which is suitable for being transported through a flow cell of a flow cytometer in a fluid stream, wherein the microparticle has a solid particle core. Furthermore, the invention relates to a method for producing a microparticle for bioanalytical investigations, which is suitable for being transported through a flow cell of a flow cytometer in a fluid stream, comprising the step of making available at least one solid particle core.

### Description of Related Art

Such microparticles are known from practice and are used for bioanalytical investigations in flow cytometers, in a size of about 2 to 10 μm. The particle cores, which are also referred to as microbeads, generally consist of polymethyl methacrylate (PMMA) or of polystyrene. Particle cores composed of transparent polystyrene can be purchased, for example, from PolyAn GmbH with domicile in Berlin, Germany. In order to achieve efficient binding of receptors, which are binding-specific for a ligand and are used in flow cytometry, for example, the particle cores are chemically modified, so as to have reactive groups such as amino groups, epoxy groups, aldehydes, for example. Receptors can be covalently or non-covalently bound to such modified particles by the user.

However, the surface of such particle cores is generally capable of only restricted bioconjugation, since there are steric or hydrophobic/hydrophilic interactions that counteract directed conjugation. If receptors are immobilized on the particle cores in the incorrect manner, problems can occur in the recognition of proteins to be bound to the ligands, such as antibodies, antigens or antisense nucleic acids, for example. As a result, the signal intensity of the optical measurement signals measured using a flow cytometer and thereby the detection sensitivity of the flow cytometry measurement is reduced.

Until now, such problems have been solved by the user by testing the different conjugation conditions. In this regard, proteins with hydrophobic regions cause particular difficulties, since most conjugation steps take place in aqueous media.

### SUMMARY OF THE INVENTION

The task therefore exists of creating a method for producing a microparticle of the type stated initially, which makes it possible to immobilize at least one receptor biomolecule having a predetermined surface occupation density on the particle core, in a simple manner, in an aqueous medium, wherein the receptor biomolecule has at least one binding site that is binding-specific for a ligand contained in a fluid to be investigated. Furthermore, the method is supposed to make it possible to apply the receptor biomolecules to the surface of the particle core at a high surface occupation density. Furthermore, the task exists of indicating such a microparticle.

This task is accomplished, with regard to the method, with the characteristics as described herein. In this regard, the method comprises the following method steps:

a) making available at least one solid particle core,
b) making available at least one nucleic acid sense biomolecule, which is binding-specific for an antisense biomolecule,
c) making available at least one functional linker group, which is suitable for binding to the surface of the particle core, and which linker group is not capable of binding to the at least one nucleic acid sense biomolecule,
d) making available at least one linear, water-soluble polymer and/or copolymer, which
has at least one first group by means of which the polymer and/or copolymer can be cross-linked by means of irradiation with optical radiation having a discrete wavelength,
has at least one second group that is suitable for binding to the at least one nucleic acid sense biomolecule, and
has at least one third group that is suitable for binding to the at least one functional linker group,
   e) producing a modified polymer and/or copolymer
by means of conjugation of the at least one nucleic acid sense biomolecule to the at least one second group of the polymer and/or copolymer, and
by means of conjugation of the at least one functional linker group to the at least one third group of the polymer and/or copolymer, and
   f) conjugating the linker group to the surface of the at least one particle core.

The task stated above is accomplished, with regard to the method, also with the characteristics as described herein. In this regard, the method comprises the following method steps:

a) making available at least one solid particle core,
b) making available at least one nucleic acid sense biomolecule that is binding-specific for an antisense biomolecule,
c) making available at least one linear, water-soluble polymer and/or copolymer, which
has at least one functional linker group that is suitable for binding to the surface of the particle core and that is not capable of binding to the at least one nucleic acid sense biomolecule,
has at least one first group by means of which the polymer and/or copolymer can be cross-linked by means of irradiation with an optical radiation having a discrete wavelength, and
has at least one second group that is suitable for binding to the at least one nucleic acid sense biomolecule,
   d) producing a modified polymer and/or copolymer by means of conjugation of the at least one nucleic acid sense biomolecule to the at least one second group of the polymer and/or copolymer, and
   e) conjugating the linker group to the surface of the at least one particle core. In this case, the linker group is therefore polymerized into the polymer and/or copolymer.

In an advantageous manner, the method makes it possible to sheathe the at least one particle core in step f) or in step e) as described herein, in a solvent, with a polymer and/or copolymer layer that has the at least one nucleic acid sense biomolecule, and anchoring it to the particle core. Since the polymer and/or copolymer layer can swell in an aqueous solution, the at least one nucleic acid sense biomolecule can be bound to the surface of the particle core at a high surface occupation density.

After coupling of the polymer and/or copolymer to the surface of the particle core and to the at least one nucleic acid sense biomolecule, at least one receptor biomolecule can be conjugated onto the nucleic acid sense biomolecule, directly or preferably indirectly, by way of the antisense biomolecule, wherein the receptor biomolecule has at least one binding site that is binding-specific for a ligand to be detected and contained in an aqueous solution. In this regard, the receptor biomolecule can be, in particular, an antibody. For binding the at least one receptor biomolecule to the nucleic acid sense biomolecule, the receptor biomolecule and the microparticle are preferably introduced into an aqueous solvent in such a manner that the receptor biomolecule comes into contact with the nucleic acid sense biomolecule (in the case of direct coupling) and/or the antisense biomolecule (in the case of indirect coupling).

In the case of a microparticle that has a plurality of nucleic acid sense biomolecules, direct or indirect binding of the receptor biomolecules to the nucleic acid sense biomolecules preferably takes place in such a manner that a receptor biomolecule is bound to essentially all the nucleic acid sense biomolecules of the microparticle. Thereby the number of receptor biomolecules situated on the surface of the particle core essentially corresponds to the number of nucleic acid sense biomolecules situated on the surface of the particle core. In one embodiment of the invention, it is actually possible that the receptor biomolecule has multiple binding sites that are binding-specific for the ligand to be detected.

Conjugation of the receptor biomolecule or of the antisense biomolecule arranged on the receptor biomolecule to the nucleic acid sense biomolecules that serve as the standard interface can be carried out by the user of the microparticles. Depending on the receptor biomolecule used and depending on the ligand for which the receptor biomolecule is binding-specific, the user has the possibility of producing modified microparticles that are adapted to a ligand to be detected, in each instance, using uniform, preferably industrially prefabricated microparticles, in a simple manner. In this regard, the number of the receptor biomolecules immobilized on the modified microparticle essentially agrees with the number of nucleic acid sense biomolecules situated on the prefabricated microparticle. The latter can be filed in a data sheet or a database, for example, by the manufacturer of the prefabricated microparticles. Thereby complicated testing of the conjugation conditions is eliminated for the user.

A polymer or copolymer that is suitable for the method according to the invention can be produced by means of polymerization or copolymerization of
dimethyl acrylamide (DMAA) and/or
Na-4-styrene sulfonate (SNa) and/or
glycidyl methacrylate (MAGE).

Multiple-functionalized copolymers can be produced from simple amino-reactive copolymers in that the desired functional components are added to the copolymer in a mixture and have the functional components of an amino group, preferably an aliphatic amino linker. Functional components are understood to be chemical groups that are suitable for covalently binding to other chemical groups, such as, for example, amino groups, thiol groups, carboxy groups, maleimides, epoxies, etc.

Using the at least one second group of the polymer and/or copolymer, the latter is coupled to the at least one nucleic acid sense biomolecule. The at least one second group can comprise, for example, at least one diene, azide (Klick Chemie), amine, thiol, active ester, epoxy, hapten (e.g., biotin), or a combination of these compounds.

Using the at least one third group of the polymer and/or copolymer, the latter is coupled to the surface of the particle core. As a result, the particle core is surrounded by a swellable polymer and/or copolymer sheath that has the nucleic acid sense biomolecules. The functional linker group is preferably not present on the at least one nucleic acid sense biomolecule, and the at least one nucleic acid sense biomolecule is preferably not capable of directly binding to the surface of the particle core.

The at least one third group can comprise, for example, at least one diene, azide (Klick Chemie), amine, thiol, active ester, epoxy, hapten (e.g., biotin), or a combination of these compounds. Binding of the third group to the surface of the particle core takes place by way of a functional linker group that is arranged between the third group and the surface of the particle core and structured in such a manner that it can bind to the third group and to the surface of the particle core. The latter result can be achieved in that the particle core is coated, on its surface, with at least one suitable coupling group, by way of which the microparticle can bind to the functional linker group. If this group contains biotin, the particle core can have a core composed of polymethyl methacrylate (PMMA) or polystyrene, which is coated with a thin layer of streptavidin, which binds to biotin upon making contact with it. The at least one third group of the polymer and/or copolymer can be identical with the second group, in terms of structure, or can differ from it.

In order to stabilize the swellable modified polymer and/or copolymer, the modified polymer and/or copolymer can be cross-linked after being applied to the particle core, by means of the at least one first group. As a result, swelling of the modified polymer and/or copolymer during hydration and therefore the swelling pressure in the modified polymer and/or copolymer are limited. Furthermore, the swellable polymer and/or copolymer allow(s) good accessibility of the ligand to the biomolecule. During swelling of the polymer and/or copolymer network, its mesh size increases, so that the ligand can better penetrate into the polymer and/or copolymer network and reach the receptor biomolecule.

It is true that a method for covalent immobilization of nucleic acid sense biomolecules on organic surfaces is already known from WO 2004/104223 A1, in which a nucleic acid sense biomolecule is provided, directly or indirectly, with at least one photo-reactive group, by way of a spacer. The reaction product obtained in this way is bound to a soluble polymer or copolymer, which is then printed onto an organic surface and covalently immobilized on this surface. However, this method is not suitable practically for the production of microparticles for bioanalytical investigations, which are supposed to be transported through a flow cell of a flow cytometer in a fluid stream, because the particle cores would clump up with the polymer or copolymer during printing. However, in a flow cytometer the microparticles must individually get into the measurement region of the energy beam or of the electrical field of the flow cytometer, in a fluid stream of an analysis medium, so that an analyte bound to the at least one nucleic acid sense biomolecule of the particle core, directly or indirectly by way of a receptor biomolecule, can be classified.

In the case of an advantageous embodiment of the method, the second and the third group are structured to be identical. As a result, the method can be carried out in a simple manner. In this case, the linker group and the nucleic acid sense biomolecule stand in competition with regard to binding to the identical second and third group. It can be set in step e) as described herein, by means of the ratio of the concentration of the linker group to the concentration of the nucleic acid sense biomolecules, how many of these identical groups bind to a linker group and how many bind to a nucleic acid sense biomolecule.

In the case of another advantageous embodiment of the method, the second and the third group are structured to be different. In this case, the second group is preferably selected in such a manner that it is not suitable for binding to the at least one functional linker group. The third group is preferably selected in such a manner that it is not suitable for binding to the at least one nucleic acid sense biomolecule.

In the case of a preferred embodiment of the invention, the modified polymer and/or copolymer is purified by means of removing unconjugated polymers, copolymers and/or unconjugated nucleic acid sense biomolecules. Purification can take place, for example, by means of molecule size exclusion centrifugation, by means of density gradient centrifugation or centrifugation. Conjugation of the linker group to the surface of the at least one particle core is preferably carried out after purification.

Furthermore, the possibility exists of first binding the modified polymer and/or copolymer to the surface of the particle cores, and afterward removing any unconjugated polymers, copolymers and/or nucleic acid sense biomolecules from the microparticle obtained in this manner, by means of molecule size exclusion centrifugation or by means of density gradient centrifugation.

By means of purification, the dynamics of the measurement signal are increased when using the microparticle produced according to the method in bioanalytical investigations for quantitative determination of an analyte contained in an aqueous solution, such as, for example, in fluorescence measurements. As a result, the detection sensitivity and measurement accuracy can be further improved.

It is advantageous if the modified polymer and/or copolymer is selected in such a manner that it has a greater affinity for the surface of the particle cores than for a solvent, and if the modified polymer and/or copolymer is brought into contact with the particle core, in the solvent, in such a manner that the surface of the particle cores is coated with the polymer and/or copolymer by means of phase extraction. In this regard, the modified polymer and/or copolymer can be cross-linked during and/or after phase extraction, by way of the first groups.

The first group preferably has at least one benzophenone group or a derivative thereof and/or at least one anthraquinone group or a derivative thereof, which serves for cross-linking of the polymer and/or copolymer. In particular, methacryloyloxy benzophenone (MABP) can be used as a benzophenone group. By means of the cross-linking, the polymer and/or copolymer becomes insoluble in water, but swells to a multiple of its dry volume in aqueous media.

In the case of an advantageous embodiment of the invention, it is provided

a) that at least one receptor antisense biomolecule conjugate is made available and conjugated to the sense biomolecule with its antisense biomolecule, and
b) that the at least one conjugate conjugated to the sense biomolecule and the polymer and/or copolymer are irradiated with the optical radiation in such a manner that
i) the conjugate is covalently bound to the polymer and/or copolymer,
ii) the polymer and/or copolymer is/are cross-linked and
iii) covalently bound to the surface of the particle core.

In this regard, the conjugate can first be brought into contact with the sense biomolecule in such a manner that the conjugate binds to the sense biomolecule by way of hydrogen bridges. As a result, it becomes possible to covalently bind the conjugate, which has been fixed in place on the polymer and/or copolymer in this manner, to the polymer and/or copolymer by means of irradiation with the optical radiation. By means of this irradiation, the polymer and/or copolymer is further cross-linked and covalently bound to the surface of the particle core. Thereby the conjugate is stabilized in the polymer and/or copolymer network obtained in this manner and on the particle core.

In the case of a preferred embodiment of the invention, the polymer and/or copolymer has at least one OH group, in particular as a component of hydroxymethyl methyl methacrylate and/or 2-methacryloyloxyethyl phosphoryl choline. In this regard, covalent coupling of the receptor biomolecule to the linear polymer and/or copolymer preferably takes place by means of exposure to optical radiation having a suitable wavelength, in particular by means of exposure to ultraviolet radiation. The receptor biomolecule is therefore covalently bound to the polymer and/or copolymer not only by way of the relatively weak hydrogen bridges between the nucleic acid sense biomolecule and the one antisense biomolecule, but, in addition, also by way of the at least one OH group. As a result, binding of the receptor biomolecule to the polymer and/or copolymer is stabilized.

Biomolecules are, for example, peptides and proteins, in particular oligopeptides having a length of 2-10 amino acids, peptides having a length of 11-80 amino acids, and proteins starting with a length of approximately 80 amino acids, PNA (Peptide Nucleic Acid), LNA (Locked Nucleic Acid). Nucleic acid sense biomolecules are, for example, DNA (Deoxyribonucleic Acid), RNA (Ribonucleic Acid), PNA (Peptide Nucleic Acid), LNA (Locked Nucleic Acid), and other nucleic acid derivatives.

It is advantageous if the polymer and/or copolymer has at least one methyl methacrylate and/or methacrylic acid group. As a result, high solubility of the polymer and/or copolymer in water is guaranteed.

In a further development of the invention, at least one first nucleic acid sense biomolecule and at least one second nucleic acid sense biomolecule that differs from the first are made available and conjugated to the polymer and/or copolymer, wherein the at least one first nucleic acid sense biomolecule is binding-specific for a first antisense biomolecule and is not capable of binding to a second antisense biomolecule that differs from the first antisense biomolecule, and wherein the at least one second nucleic acid sense biomolecule is binding-specific for the second antisense biomolecule and is not capable of binding to the first antisense biomolecule. The microparticle produced according to the method can then be used in a flow cytometer for multi-channel or multi-dimensional measurements in which the concentrations of multiple analytes contained in an aqueous solution are measured simultaneously, using the same microparticle.

A copolymer can be produced, for example, from DMAA, coMABP, coMAGE. This copolymer has a preferred size (MW) of 50 to 500 kilodaltons, particularly preferably 150 to 300 kilodaltons. In this regard, MABP contents of 1% to 7% (mol%), in particular of 2% to 5% (mol %), and PGMA contents of 1% to 7% (mol %), in particular of 2% to 5% (mol %) are preferred.

For producing biofunctionalized copolymers, the copolymer (DMAA, coMABP, coMAGE) can be dissolved in PBS buffer, so as to produce a solution of 10 mg/ml (1% w/v). In the case of higher MABP and MAGE contents, it can be necessary to add a solubilizer, for example ethanol in a concentration of 1 to 10 mol %.

The desired functionalizations are then added to the solution. In order to bind to the epoxy group of the MAGE, the functionalizations must have a primary amino group (or thio group). Thus, a mixture of 20 mol % amino biotin, 40 mol % C6-amino DNA1 and 40 mol % C6-amino DNA2 (mol %) can be used, so as to produce a biotinylated copolymer having two different oligonucleotide modifications. In this regard, “DNA1” refers to a first deoxyribonucleic acid and “DNA2” refers to a second deoxyribonucleic acid, which differs from DNA1.

Since both the amino biotin and the oligonucleotides have a molecular weight of less than 100 kilodaltons, components that might not have reacted can be removed by means of chromatography or size exclusion centrifugation. A polymer having 20 PGMA groups now has 4 biotin groups, 8 times DNA1, and 8 times DNA2, after the modification. If one wishes to quantify the DNA groups, one can provide the DNA with a fluorescence marker. In this regard, the DNA1 and the DNA2 preferably have different markers having distinguishable emission wavelengths (for example Cy3 and Cy5).

Nucleic acid sense biomolecules: peptides and proteins, in particular oligopeptides having a length of 2-10 amino acids, peptides having a length of 11-80 amino acids, and proteins starting from a length of approximately 80 amino acids, PNA (Peptide Nucleic Acid), LNA (Locked Nucleic Acid).

With regard to the microparticle of the type stated initially, the task stated above is accomplished with the characteristics as described herein. These provide that a layer of water-soluble conjugate is arranged on the particle core, which conjugate comprises at least one linear polymer and/or copolymer, which

a) has at least one first group by means of which the polymer and/or copolymer can be cross-linked by means of irradiation using optical radiation having a discrete wavelength, and can be covalently bound to the surface of the particle core as well as to a receptor antisense biomolecule conjugate,
b) has at least one second group to which the at least one nucleic acid sense biomolecule is conjugated, and
c) has at least one third group that is bound to the surface of the particle core by way of a functional linker group, wherein the linker group is not capable of binding to the at least one nucleic acid sense biomolecule.

The particle core is therefore coated with a polymer and/or copolymer sheath, and preferably sheathed by it, by way of which sheath the at least one nucleic acid sense biomolecule is immobilized on the particle core. Due to the swellability, the polymer and/or copolymer network becomes easily permeable for the ligand molecules. Due to the swellability of the polymer and/or copolymer sheath with water, the particle cores according to the invention allow a great surface occupation density of the surface of the particle core with the nucleic acid sense biomolecules. As a result, binding of an analyte contained in an aqueous solution to the nucleic acid sense biomolecules or a receptor biomolecule specifically bound to them, using the particle core, can be detected with correspondingly great detection sensitivity and measurement accuracy in a flow cytometer.

In the case of an advantageous embodiment of the microparticle, the second and the third group are structured to be identical. As a result, the microparticle can be produced in a simple manner.

In the case of another embodiment of the microparticle, the second and the third group are structured to be different. In this regard, the second group is preferably not suitable for binding to the at least one functional linker group, and the third group is preferably not suitable for binding to the at least one nucleic acid sense biomolecule.

The first group of the microparticle preferably comprises at least one benzophenone group or a derivative thereof and/or at least one anthraquinone group or a derivative thereof.

In the case of an advantageous embodiment of the microparticle according to the invention, at least one antisense biomolecule of a receptor antisense biomolecule conjugate is conjugated to the sense biomolecule, wherein

i) the conjugate is covalently bound to the polymer and/or copolymer,
ii) the polymer and/or copolymer is cross-linked, and
iii) covalently bound to the surface of the particle core.

By means of the covalent bonds, the conjugate is stabilized in the cross-linked polymer and/or copolymer.

The polymer and/or copolymer of the microparticle can have at least one OH group, in particular as a component of hydroxymethyl methyl methacrylate and/or 2-methacryloyloxy ethyl phosphoryl choline.

In the case of a preferred embodiment of the particle core according to the invention, at least one first nucleic acid sense biomolecule and at least one second nucleic acid sense biomolecule that differs from the first are conjugated to the polymer and/or copolymer, wherein the at least one first nucleic acid sense biomolecule is binding-specific for a first antisense biomolecule and not capable of binding to a second antisense biomolecule that differs from the first antisense biomolecule, and wherein the at least one second nucleic acid sense biomolecule is binding-specific for the second antisense biomolecule and not capable of binding to the first antisense biomolecule.

The microparticle makes it possible to simultaneously measure multiple analytes contained in an aqueous solution, in particular using a flow cytometer.

It is advantageous if the layer composed of the water-soluble nucleic acid sense biomolecule polymer conjugate and/or the nucleic acid sense biomolecule copolymer conjugate arranged on the solid particle core has a thickness, orthogonal to the surface of the particle core, in the swelled state, of at least 20 nm, in particular at least 100 nm, and preferably at least 150 nm. This allows a great surface occupation density of the surface with the nucleic acid sense biomolecules.

The dimensions of the particle core can amount to 0.1 μm to 200 μm, possibly 0.5 μm to 50 μm, in particular 1 μm to 20 μm, and preferably 5 μm to 7 μm. The particle core is preferably configured to be essentially spherical.

It should also be mentioned that in the case of a preferred embodiment of the invention, the nucleic acid sense biomolecules are marked with a marker for quantification. The marker can be, in particular, an optical marker, such as Cy3 or Cy5, for example. As a result, the user of the microparticle has the possibility of measuring the amount of the nucleic acid sense biomolecules situated on the microparticle, and thereby the number of antisense biomolecules that can bind to the microparticle, in a known manner.

In the following, an exemplary embodiment of the invention will be explained in greater detail.

### 1. Production of a Copolymer

A copolymer is produced by means of copolymerization of methacryloyl benzophenone (MABP), glycidyl methacrylate (MAGE), and dimethyl acrylamide (DMAA) in a ratio of 2:2:96, in a solvent. Chloroform is used as the solvent. The copolymerization of the stated monomers is initiated by means of adding 1% azo-bis-isobutyronitrile (AIBN) to the solution. The finished copolymer is separated by means of precipitation with diethyl ether, and freed of solvent residues by means of freeze-drying.

A photo-reactive first chemical group is contained in the monomer MABP, which is structured as a benzophenone group. This group is built into the copolymer during copolymerization. Using the first group, the copolymer can be cross-linked in a later step, by means of irradiation with optical radiation.

The glycidyl methacrylate (MAGE) furthermore has at least one chemically reactive group in which the epoxy group is present as a glycidyl group, which can covalently bind to amino groups. The individual latent groups are identical, in terms of their structure, and become, by means of functionalization, in each instance, either a second chemical group that is suitable for binding to a first or second nucleic acid sense molecule, defined in greater detail in Section 2 a) or b), or a third chemical group that is suitable for binding to a functional linker group, defined in greater detail in Section 2 c).

Functionalization of the identical groups takes place, in the case of the second chemical group, by means of a chemical reaction of the glycidyl group with the first or second nucleic acid sense molecule, and, in the case of the third chemical group, by means of a chemical reaction of the glycidyl group with the functional linker group.

### 2. Modification of the Copolymer

The copolymer from Section 1, DMAA co 2% MABP co 2% MAGE, is dissolved in nuclease-free water in a concentration of 0.1 mg/ml. Due to the hydrolysis sensitivity of the MAGE, the solution is immediately used further. For this purpose, the following batch is produced:

a) 5.55 μl 100 μmol/l 5′-amino-modified DNA (oligonucleotide 1, also called first nucleic acid sense biomolecule hereinafter);
b) 5.55 μl 100 μmol/l 5′-amino-modified DNA (oligonucleotide 2, also called second nucleic acid sense biomolecule hereinafter);
c) 4.20 μl 0.1 mg/ml EZ-Link™ amine-PEG2 biotin, available from the company ThermoFischerScientific™;
d) 25 μl 10× phosphate-buffered saline solution (PBS);
e) 4.7 μl nuclease-free water.

The batch is mixed up in nuclease-free water. In this process, the first and second nucleic acid sense biomolecule are each added to the aqueous solution in a five-times molar excess, and the linker is added in a 10-times molar excess, with reference to the unmodified copolymer, in each instance.

The first and second nucleic acid sense biomolecules each have an amino group that binds to one of the second groups of the copolymer, in the phosphate-buffered saline solution. The nucleic acid sense biomolecules do not contain a biotin group and are not capable of directly binding to the surface of the particle core.

The EZ-Link™ amine-PEG2 biotin serves as a functional linker group for coupling the modified copolymer to the surface of the particle core. The amino group of the EZ-Link™ amine-PEG2 biotin binds to a third group of the copolymer, in each instance, in the phosphate-buffered saline solution. The functional linker group is selected in such a manner that it is not capable of binding to the first and second nucleic acid sense biomolecules.

The batch is incubated at +4° C. for 36 hours, and the components not incorporated into the copolymer are removed by means of size exclusion centrifugation (30 kilodaltons). The volume is subsequently adjusted back to 50 μl by means of adding PBS.

Thereby a modified copolymer is obtained, which contains the copolymer described in Section 1, to which the first and second nucleic acid sense biomolecule and the EZ-Link™ amine-PEG2 biotin are bound.

3. Binding the Modified copolymer to Particle Cores

Spherical particle cores composed of polymethyl methacrylate (PMMA), coated with streptavidin, having a diameter of 6.8 μm, are made available. The particle cores can be purchased from PolyAn GmbH in Berlin, Germany.

5 μl of the batch from Section 2 are thoroughly mixed with 5000 of the particle cores in 45 μl PBS, by means of vortexing with a duration of 30 seconds. In this process, the functional linker group conjugated to the third group of the copolymer binds to the streptavidin of the particle cores. After 30 minutes, the particle cores are separated from the non-bound copolymer by means of centrifugation.

4. Conjugation of Antibodies with Antisense DNA

Monoclonal first and second antibodies, such as anti-CRP, for example, are oxidized with potassium periodate, so that at the glycosylation of the antibodies, an aldehyde function occurs, in each instance. For this purpose, 10 mg/ml antibodies are dissolved in 150 mM sodium chloride solution pH 7.2. 10 μl of a 0.1 molar sodium periodate solution are added to 100 μl of the antibody solution and mixed well by means of vortexing. The mixture obtained in this manner is incubated for 30 minutes at room temperature, with light excluded.

Subsequently, the sodium periodate is removed by means of size exclusion centrifugation. Then the antibody is incorporated into 0.2 M sodium bicarbonate buffer pH 9.6, so as to obtain 10 mg/ml antibody concentration once again.

This antibody with an aldehyde function is then incubated with an amino-modified antisense oligonucleotide. For this purpose, the oligonucleotide is added to the antibody in a 3-times molar excess. 100 μl 200 μmol/l in sodium bicarbonate buffer pH 9.6 are added to 100 μl 67 μmol/l antibody and incubated for 24 h at +4° C. Subsequently, non-bound oligonucleotide is removed by means of size exclusion centrifugation. The volume is adjusted to 100 μl with 1× PBS.

The first antibodies each have a first antisense group, which groups are binding-specific for the first nucleic acid sense biomolecules. The second antibodies each have a second antisense group, which groups are binding-specific for the second nucleic acid sense biomolecules.

Furthermore, the first antibodies each have at least one first binding site that is binding-specific for a first analyte. In a corresponding manner, the second antibodies each have at least one second binding site that is binding-specific for a second analyte.

5. Binding of the Antisense Protein Conjugate to the Particle cores

The 5000 particle cores (10 μl, 1× PBS) from Section 3, coated with DNA copolymer, are conjugated with 5 μl of molecules of the first antibody and with 5 μl of molecules of the second antibody from Example 4. In this process, the first antibodies each bind to a first nucleic acid sense biomolecule, and the second antibodies each bind to a second nucleic acid sense biomolecule. The conjugate obtained in this manner is incubated at 20° C. (1 h in 150 mM sodium phosphate buffer pH 7.2). Subsequently, the microparticles are removed by means of centrifugation of non-bound antibody, and transferred to a quartz cuvette.

In the quartz cuvette, the microparticles are dispersed well by means of shaking, and then directly exposed to UV light (wavelength 254 nm, 1 J/cm² energy). In order to prevent cross-linking or clumping of multiple microparticles, the cuvette containing the particle cores is placed in a compact, commercially available ultrasound device and agitated by means of ultrasound.

Exposure takes place in a STRATALINKER® 2400, which is large enough to hold the ultrasound device in the inner irradiation space. During this process, the antibodies are covalently bound to the modified copolymer. Furthermore, the copolymer is cross-linked with itself and with the particle core.

The microparticles obtained in this manner, containing the antibodies and the cross-linked, modified copolymer, can be used for bioanalytical investigations.

1. A method for producing a microparticle for bioanalytical investigations, which is suitable for being transported through a flow cell of a flow cytometer in a fluid stream, comprising the step of:
a) making available at least one solid particle core, characterized by the following further steps:
b) making available at least one nucleic acid sense biomolecule, which is binding-specific for an antisense biomolecule,
c) making available at least one functional linker group, which is suitable for binding to the surface of the particle core, and which linker group is not capable of binding to the at least one nucleic acid sense biomolecule,
d) making available at least one linear, water-soluble polymer and/or copolymer, which
has at least one first group by means of which the polymer and/or copolymer can be cross-linked by means of irradiation with optical radiation having a discrete wavelength and can be covalently bound to the surface of the particle core as well as to a receptor antisense biomolecule conjugate,
has at least one second group that is suitable for binding to the at least one nucleic acid sense biomolecule, and
has at least one third group that is suitable for binding to the at least one functional linker group,
e) producing a modified polymer and/or copolymer
by means of conjugation of the at least one nucleic acid sense biomolecule to the at least one second group of the polymer and/or copolymer, and
by means of conjugation of the at least one functional linker group to the at least one third group of the polymer and/or copolymer, and
f) conjugating the linker group to the surface of the at least one particle core.

2. The method according to claim 1, wherein the second and the third group are structured to be identical.

3. The method according to claim 1, wherein the second and the third group are structured to be different.

4. A method for producing a microparticle for bioanalytical investigations, which is suitable for being transported through a flow cell of a flow cytometer in a fluid stream, comprising the step of:
a) making available at least one solid particle core,
characterized by the following further steps:
b) making available at least one nucleic acid sense biomolecule that is binding-specific for an antisense biomolecule,
c) making available at least one linear, water-soluble polymer and/or copolymer that
has at least one functional linker group that is suitable for binding to the surface of the particle core, and that is not capable of binding to the at least one nucleic acid sense biomolecule,
has at least one first group, by means of which the polymer and/or copolymer can be cross-linked by means of irradiation with optical radiation having a discrete wavelength, and
has at least one second group that is suitable for binding to the at least one nucleic acid sense biomolecule,
d) producing a modified polymer and/or copolymer, by means of conjugation of the at least one nucleic acid sense biomolecule to the at least one second group of the polymer and/or copolymer, and
e) conjugating the linker group to the surface of the at least one particle core.

5. The method according to claim 1, wherein the modified polymer and/or copolymer is purified by means of removing unconjugated polymers, copolymers and/or unconjugated nucleic acid sense biomolecules, preferably before the linker group is conjugated to the surface of the at least one particle core.

6. The method according to claim 1, wherein the modified polymer and/or copolymer is selected in such a manner that it has a greater affinity for the surface of the particle core than for a solvent, and that the modified polymer and/or copolymer is brought into contact with the particle core, in the solvent, in such a manner that the surface of the particle core is coated with the polymer and/or copolymer by means of phase extraction.

7. The method according to claim 1, wherein the first group comprises at least one benzophenone group or a derivative thereof and/or at least one anthraquinone group or a derivative thereof.

8. The method according to claim 1, wherein
a) that at least one receptor antisense biomolecule conjugate is made available and conjugated to the sense biomolecule with its antisense biomolecule, and
b) that the at least one conjugate conjugated to the sense biomolecule and the polymer and/or copolymer is/are irradiated with the optical radiation, in such a manner that
i) the conjugate is covalently bound to the polymer and/or copolymer,
ii) the polymer and/or copolymer is cross-linked and
iii) covalently bound to the surface of the particle core.

9. The method according to claim 1, wherein the polymer and/or copolymer has at least one OH group, in particular as a component of hydroxymethyl methyl methacrylate and/or 2-methacryloyloxy ethyl phosphoryl choline.

10. The method according to claim 1, wherein at least one first nucleic acid sense biomolecule and at least one second nucleic acid sense biomolecule that differs from the first are made available and conjugated to the polymer and/or copolymer, that the at least one first nucleic acid sense biomolecule is binding-specific for a first antisense biomolecule and not capable of binding to a second antisense biomolecule, which is different from the first antisense biomolecule, and that the at least one second nucleic acid sense biomolecule is binding-specific for the second antisense biomolecule and not capable of binding to the first antisense biomolecule.

11. A microparticle for bioanalytical investigations, which is suitable for being transported through a flow cell of a flow cytometer in a fluid stream, wherein the microparticle has a solid particle core and at least one nucleic acid sense biomolecule that is binding-specific for an antisense biomolecule, wherein a layer composed of a water-soluble conjugate is arranged on the particle core, which layer comprises at least one linear polymer and/or copolymer, which
a) has at least one first group, by means of which the polymer and/or copolymer can be cross-linked by means of irradiation with optical radiation having a discrete wavelength, and can be covalently bound to the surface of the particle core as well as to a receptor antisense biomolecule conjugate,
b) has at least one second group to which the at least one nucleic acid sense biomolecule is conjugated, and
c) has at least one third group that is bound to the surface of the particle core by way of a functional linker group, wherein the linker group is not capable of binding to the at least one nucleic acid sense biomolecule.

12. The microparticle according to claim 11, wherein the second and the third group are structured to be identical.

13. The microparticle according to claim 11, wherein the second and the third group are structured to be different.

14. The microparticle according to claim 11, wherein the first group comprises a benzophenone group or a derivative thereof and/or at least one anthraquinone group or a derivative thereof.

15. The microparticle according to claim 11, wherein at least one antisense biomolecule of a receptor antisense biomolecule conjugate is conjugated to the sense biomolecule, and
i) that the conjugate is covalently bound to the polymer and/or copolymer,
ii) the polymer and/or copolymer is cross-linked and
iii) covalently bound to the surface of the particle core.

16. The microparticle according to claim 11, wherein the polymer and/or copolymer has at least one OH-group, in particular as a component of hydroxymethyl methyl methacrylate and/or 2-methacryloyloxy ethyl phosphoryl choline.

17. The microparticle according to claim 11, wherein at least one first nucleic acid sense biomolecule and at least one second nucleic acid sense biomolecule that differs from the first are conjugated to the polymer and/or copolymer, that the at least one first nucleic acid sense biomolecule is binding-specific for a first antisense biomolecule and not capable of binding to a second antisense biomolecule that differs from the first antisense biomolecule, and that the at least one second nucleic acid sense biomolecule is binding-specific for the second antisense biomolecule and not capable of binding to the first antisense biomolecule.

18. The microparticle according to claim 11, wherein the layer composed of the water-soluble nucleic acid sense biomolecule polymer and/or copolymer conjugate arranged on the solid particle core has a thickness, orthogonal to the surface of the particle core, in the swollen state, of at least 20 nm, in particular at least 100 nm, and preferably at least 150 nm.
